(19) United States
(12) Patent Application Publication
(10) Pub. No.: US 2006/0292698 A1
(43) Pub. Date: Dec. 28, 2006

(54) SPECTROSCOPIC METHODS FOR DETECTING AND IDENTIFYING CHELATES

(76) Inventors: Jennifer W. Hartle, Harrisville, UT (US); Clayton Ericson, Morgan, UT (US); Robert C. Thompson, Morgan, UT (US)

Correspondence Address: BRINKS HOFER GILSON & LIONE;UTAH OFFICE, 299 SO. MAIN, SUITE 1300, SALT LAKE CITY, UT, 84111-2241, US

(21) Appl. No.: 11/426,553

(22) Filed: Jun. 26, 2006

Related U.S. Application Data
(60) Provisional application No. 60/693,709, filed on Jun. 24, 2005.

(51) Int. Cl. G01N 33/20

(52) U.S. Cl. 436/74

(57) ABSTRACT

Methods are provided for characterizing metal-ligand chelates using Raman spectroscopy.

### CROSS REFERENCE TO RELATED APPLICATION

This application claims benefit of the filing date of U.S. Provisional Patent Application No. 60/693,709, filed Jun. 24, 2005, the disclosure of which is incorporated, in its entirety, by this reference.

### TECHNICAL FIELD OF THE INVENTION

The present invention relates to spectroscopic methods that can be used to qualitatively and quantitatively measure chelation of organic compounds with various cationic minerals such as, amino acid compounds with various minerals.

### BACKGROUND OF THE INVENTION

Chelates are generally produced by the reaction or association of a ligand with a metal cation, resulting in a complex. Amino acid chelates may be made by the reaction of an α-amino acid and metal ion typically, but not necessarily, having a valence of two or more to form a ring structure. In such a reaction, the positive electrical charge of the metal ion is neutralized or delocalized by the electrons available through the carboxylate and or free amino groups of the α-amino acid. The structure, chemistry and bioavailability of amino acid chelates is well documented in the literature, e.g. Ashmead et al., Chelated Mineral Nutrition, (1982), Chas. C. Thomas Publishers, Springfield, Ill.; Ashmead et al., Intestinal Absorption of Metal Ions, (1985), Chas. C. Thomas Publishers, Springfield, Ill.; Ashmead et al., Foliar Feeding of Plants with Amino Acid Chelates, (1986), Noyes Publications, Park Ridge, N.J.

One advantage of amino acid chelates in the field of mineral nutrition is that they are readily absorbed in the gut and mucosal cells by means of active transport. Chelates enable minerals to be absorbed in biological processes along with amino acids as a single unit utilizing the amino acids as carrier molecules. Therefore, the problems associated with the competition of ions for active sites and the suppression of specific nutritive mineral elements by others can be avoided. This is especially true for compounds such as iron sulfates that are currently delivered in relatively large quantities in order for the body to absorb an effective amount. Controlled delivery of nutritional minerals is advantageous because large quantities of those minerals often cause nausea and other discomforts as well as create an undesirable taste.

Since metal amino acid chelates can serve as a delivery means for mineral supplements, there is a growing need for methods of characterizing chelates, such as amino acid chelates. Government regulation and guidelines associated with nutritional manufacturing are prompting the development of techniques to quantify a variety of ingredients. Presently, there is no accepted method for the detection, identification and quantification of metal amino acid chelates by the United States Pharmacopeial Convention (USP), Association of Analytical Communities (AOAC), or the United States Food and Drug Administration (FDA). A technique that can detect, identify and quantify chelates, and specifically metal amino acid chelates, is highly desirable since that technique could be used in the nutritional and feedstock industries to attain reliable comparisons and standards for uniform treatment of nutritional supplements and ingredients. Furthermore, a method of detecting metal chelates would be useful in a variety of contexts including analyzing waste water treatment, removing heavy or radioactive elements from waste streams, and characterizing new and novel chelates.

### SUMMARY OF THE INVENTION

The present invention is directed to methods of characterizing chelates. In one aspect, a method provides for assaying for the presence or absence of a metal-ligand chelate in a sample comprising:

irradiating with electromagnetic energy a sample suspected of containing one or more of a free ligand and a metal-ligand chelate;

obtaining a Raman electromagnetic spectrum of the sample;

analyzing the Raman electromagnetic spectrum to detect one or more of a free metal, the free ligand, and the metal-ligand chelate, the presence or absence of which is indicative of the metal-ligand chelate.

In another aspect, a method provides detecting for the presence or absence of a metal-ligand chelate in a sample comprising:

irradiating with electromagnetic energy a sample containing a ligand;

obtaining a first Raman electromagnetic spectrum of the ligand;

contacting and/or reacting the ligand in the sample with a metal;

obtaining a second Raman electromagnetic spectrum;

comparing the first Raman electromagnetic spectrum with the second Raman electromagnetic spectrum to detect a change.

In still another aspect, a method provides for monitoring chelation synthesis reactions comprising:

irradiating a sample containing a free metal and a free ligand with electromagnetic energy;

obtaining a Raman spectrum of the sample;

reacting the free metal with the free ligand to form a metal-ligand chelate;

analyzing the Raman spectrum to detect one or more of the free metal, the free ligand, and metal-ligand chelate, the presence or absence of which is indicative of the progress of chelation synthesis.

In some embodiments, electromagnetic energy may be supplied by a laser.

In some embodiments, a method includes a metal selected from the group consisting of: boron, calcium, chromium, cobalt, copper, iron, magnesium, manganese, potassium, selenium, vanadium and zinc, and a ligand selected from the group consisting of: alanine, aspartic acid, cysteine, glutamic acid, glycine, histidine, isoleucine, leucine, lysine, methionine, phenylalanine, serine, threonine, tryptophan, tyrosine and valine.

In some embodiments, a method includes a ligand is selected from the group consisting of: alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine.

In some embodiments, a method is further characterized by at least one of the first and second Raman electromagnetic spectra having a wavelength peak corresponding to one or more functional groups selected from the group consisting of: amine, hydroxyl, thiol, and carboxylic acid.

In some embodiments, a method further includes

obtaining a first IR electromagnetic spectrum of the sample;

obtaining a second IR electromagnetic spectrum;

comparing the first IR electromagnetic spectrum with the second IR electromagnetic spectrum to detect a change.

In some embodiments, a method further includes analyzing the Raman and IR spectral changes to characterize a chelate.

In some embodiments, a method further includes calculating a reaction rate.

In some embodiments, a method is further characterized in that a free metal and a free ligand are contacted at a pH of between about 3 and about 10.

In some embodiments, a method is further characterized in that an Raman spectrum is obtained with a sample at a pH of between about 3 and about 10.

In some embodiments, a method is further characterized in that an IR spectrum is obtained with a sample at a pH of between about 3 and about 10.

### BRIEF DESCRIPTION OF THE DRAWINGS

FIG. 1 is a plot of Raman scatter intensity at 1324 cm⁻¹ for sequential measurements of the reaction of zinc oxide with glycine and depicts the R-squared fit for first ordered kinetic reaction.

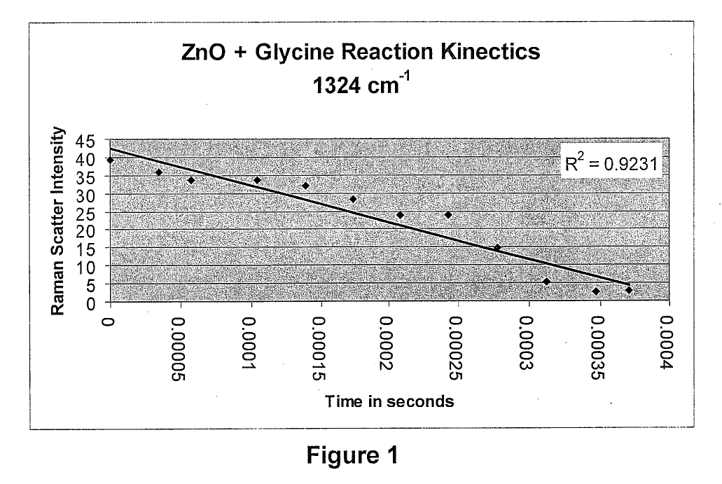

FIG. 2 is a plot of Raman scatter intensity at 1136 cm⁻¹ for sequential measurements of the reaction of zinc oxide with glycine and depicts the R-squared fit for first ordered kinetic reaction.

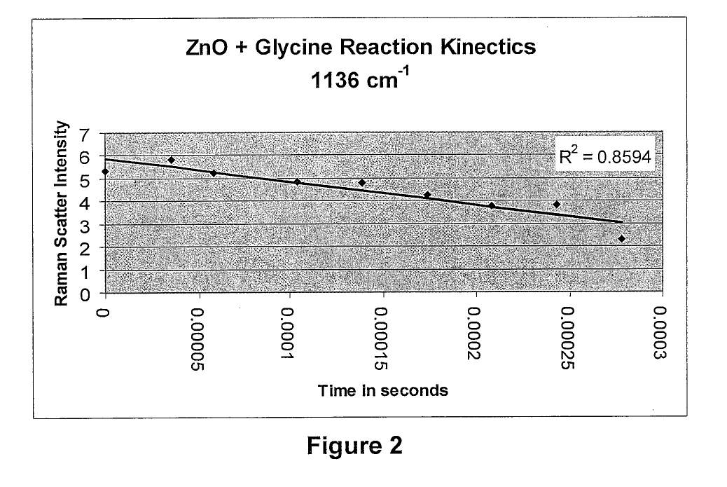

FIG. 3 is a plot of Raman scatter intensity at 1107 cm⁻¹ for sequential measurements of the reaction of zinc oxide with glycine and depicts the R-squared fit for first ordered kinetic reaction.

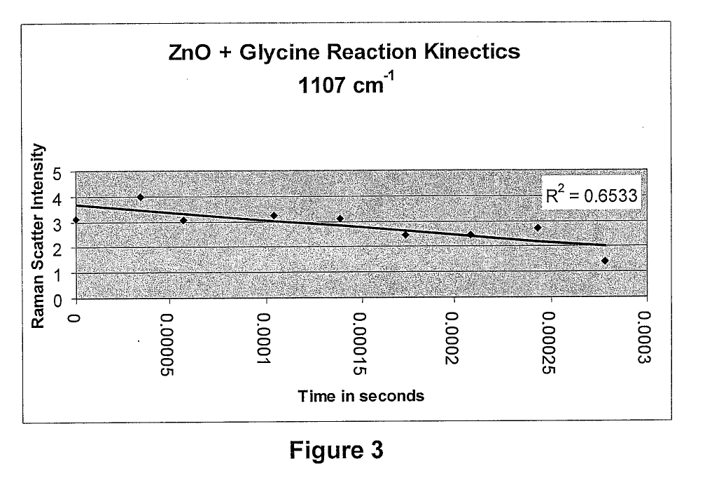

FIG. 4 is a plot of Raman scatter intensity at 1035 cm⁻¹ for sequential measurements of the reaction of zinc oxide with glycine and depicts the R-squared fit for first ordered kinetic reaction.

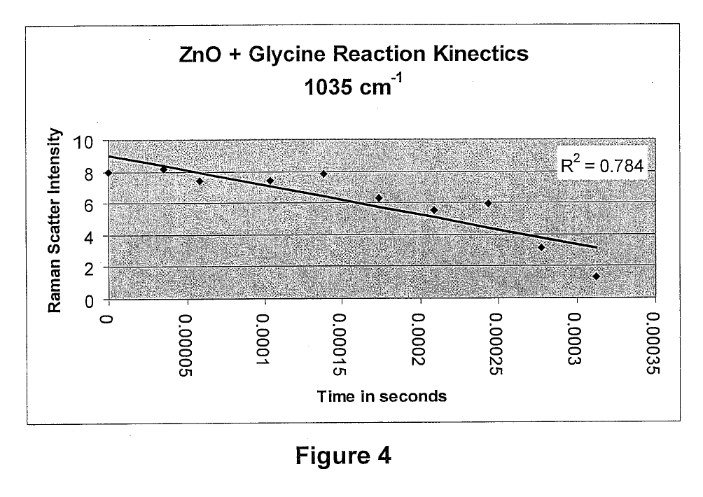

FIG. 5 is a plot of Raman scatter intensity at 892 cm⁻¹ for sequential measurements of the reaction of zinc oxide with glycine and depicts the R-squared fit for first ordered kinetic reaction.

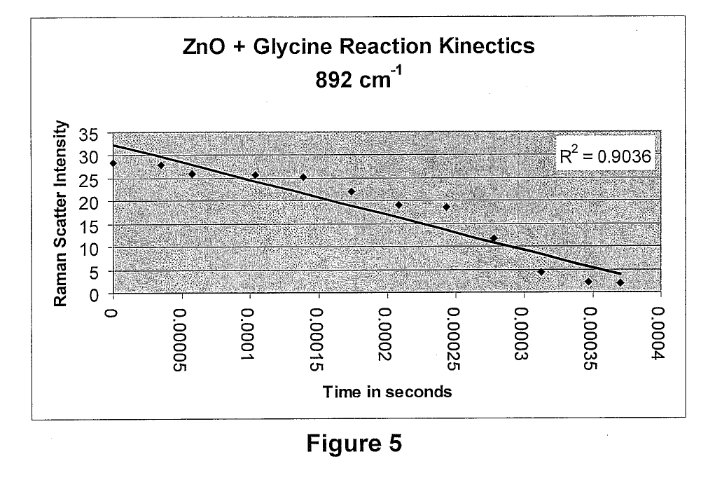

### DETAILED DESCRIPTION OF THE INVENTION

Unless defined otherwise, technical and scientific terms used herein have the same meaning as commonly understood by one of ordinary skill in the art to which this invention belongs. Units, prefixes, and symbols may be denoted in their SI accepted form. Unless otherwise indicated. Numeric ranges recited herein are inclusive of the numbers defining the range and include and are supportive of each integer within the defined range. Unless otherwise noted, the terms “a” or “an” are to be construed as meaning “at least one of.” The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose. One skilled in the art will recognize many methods and materials similar or equivalent to those described herein, which could be used in the practice of the present invention. The present invention is in no way limited to the methods and materials described. For purposes of the present invention, the following terms are defined below.

The term “chelate” as used herein means a molecular entity made up of a central metal associated with at least one bidentate ligand and optionally associated with one or more mono- or multi-dentate ligands. In the interaction between the central metal and any of the ligands, the bonds between the ligand and the central metal can include covalent bonds, ionic bonds, and/or coordinate covalent bonds.

As applied in the field of mineral nutrition, there are at least two chelated products which are commercially utilized. The first is referred to as a “metal proteinate.” The American Association of Feed Control Officials (AAFCO) has defined a “metal proteinate” as the product resulting from the chelation of a soluble salt with amino acids and/or partially hydrolyzed protein. Such products are referred to as the specific metal proteinate, e.g., copper proteinate, zinc proteinate, etc. These metal proteinates also include at least one chelate ring.

Proteinates can be formed using dipeptides, tripeptides, tetrapeptides, polypeptides. Larger ligands have a molecular weight that is too great for direct assimilation of the chelate formed. Generally, peptide ligands will be derived by the hydrolysis of protein. However, peptides prepared by conventional synthetic techniques or genetic engineering can also be used. When a ligand is a di- or tripeptide, a radical of the formula [C(O)CHR₁NH]_{g}H will replace one of the hydrogens attached to the nitrogen atom in Formula 1. R₁, as defined in Formula 1, can be H, or the residue of any other naturally occurring amino acid and g can be an integer of 1, 2 or 3. When g is 1 the ligand will be a dipeptide, when g is 2 the ligand will be a tripeptide and so forth. Amino acid chelates can also include cyclic peptides ligands such as those peptides which can act as cryptands.

An “amino acid chelate” as used herein means the product resulting from the reaction of a metal or metal ion from a soluble metal salt with one or more amino acids having a mole ratio of from 1:1 to 1:4, or, in particular embodiments, having a mole ratio 1:2, moles of metal to moles of amino acids, to form coordinate covalent bonds. The average weight of the hydrolyzed amino acids is approximately 150 and the resulting molecular weight of the chelate will typically not exceed a molecular weight of about 800 amu and more frequently less than about 1000 amu. The chelate products can be identified by the specific metal forming the chelate (e.g., iron amino acid chelate, copper amino acid chelate, etc.) An amino acid chelate may be represented at a ligand to metal molar ratio of 2:1 according to Formula 1 as follows: 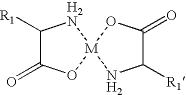
where R₁ and R₁′ are organic radicals, substituents or functional groups. R₁ and R₁′ can be the same or different.

In the above formula, the dashed lines can represent coordinate covalent bonds, covalent bonds, and/or ionic bonds. Further, when R₁ is H, the chelating agent is an amino acid, glycine that is the simplest of the α-amino acids. However, R₁ could be representative of any other side chain. Where the chelating agent is one of the naturally occurring α-amino acids, the R₁ side chains have been described as aliphatic which includes but is not limited to alanine, glycine, isoleucine, leucine, proline, and valine; aromatic which includes but is not limited to phenylalanine, tryptophan, tyrosine; acidic which includes but is not limited to aspartic acid, and glutamic acid; basic which includes but is not limited to arginine, histidine, and lysine; hydroxylic which includes but is not limited to serine, and threonine; sulfur-containing which includes but is not limited to cysteine, and methionine; amidic (containing amide group) which includes but is not limited to asparagine, and glutamine. R₁ could also be representative of any other side chain resulting in any of the non-natural occurring amino acids. Many of the amino acids have the same configuration for the positioning of the carboxylic acid oxygens and the α-amino nitrogen with respect to the metal ion. In other words, the chelate ring can be defined by the same atoms in each instance, even though the R₁ side chain group may vary. In some embodiments, amino acids with non-nucleophilic R groups include alanine, glycine, histidine, isoleucine, leucine, methionine, phenylalanine, serine, tryptophan, and valine.

The term “chelate ring” as used herein means the atoms of the ligand and central metal which form a heterocyclic ring with the metal as the closing member. In the interaction between the central metal and a multidentate ligand, one or more chelate rings of from 3 to 8 members can exist. The chelate ring can be of from 5 to 6 members.

The term “ligand” as used herein means a molecular group that is associated with a central metal atom. The ligand can be any ligand capable of forming a chelate with a metal. The terms monodentate, bidentate (or didentate), tridentate, tetradentate, and multidentate are used to indicate the number of potential binding sites of the ligand. For example, a carboxylic acid can be a bidentate or other multidentate ligand because it has at least two binding sites, the carboxyl oxygen and hydroxyloxygen. An amino acid can have at least two binding sites and many amino acids will have multiple binding sites including the amino nitrogen and the carboxyl oxygen and hydroxyloxygen atoms of a carboxylic acid functional group. When the side chain of the amino acid has one or more heteroatoms, the side chain may also present additional binding sites.

Examples of ligands include those with primary or secondary amines and more preferred ligands are those with primary amines. Other examples of ligands are those with primary or secondary amines and a carboxylic acid, each of which is α to a common carbon atom. Ligands can include those with primary and/or secondary amines. Ligands can include amino acids with primary amines. Ligands can also include primary or secondary amines each with a carboxylic acid β to the primary or secondary amine. Representative ligands include but are not limited to the α-amino acids which include the selected from the naturally occurring amino acids commonly found in biological structures including alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In some examples, the amino acid ligands may be selected from alanine, glycine, histidine, isoleucine, leucine, methionine, phenylalanine, serine, tryptophan, and valine. Other ligands include the amino acids 4-hydroxyproline, 5-hydroxylysine, homoserine, homocysteine, ornithine, β-alanine, γ-aminobutyric acid (GABA), statine, ornithine, and statin. In other embodiments, the amino acid is selected from the non-natural amino acids. In some embodiments, the amino acid is selected from the aliphatic naturally occurring amino acids selected from alanine, glycine, isoleucine, leucine, proline, and valine. Amino acids ligands can be the L-amino acids, the D-amino acids, or a racemic mixture. In some embodiments, the amino acids are the L-amino acids.

The term “metal” as used herein means any alkaline, alkaline earth, transition, and rare earth, basic, and semi-metals which can coordinate with a ligand. Metal can include nutritional minerals. Representative metals include the transition, lanthanide, and actinide metals. In some embodiments, the metal has d-orbitals capable of interacting with a ligand. In some embodiments, metals are selected from boron, calcium, chromium, cobalt, copper, iron, magnesium, manganese, molybdenum, potassium, selenium, vanadium, and zinc.

The term “nutritionally acceptable metal” as used herein means metals that are known to be needed by living organisms, particularly plants and mammals, including humans. Metals such as boron, calcium, chromium, cobalt, copper, iron, magnesium, manganese, potassium, selenium, vanadium, and zinc, among others, are examples of nutritionally acceptable metals.

The term “polyligated” as used herein means 2 or more ligands associated or bound to a metal.

The terms “hydrate” or “n-hydrate” as used herein means a molecular entity with some degree of hydration, where n is an integer representing the number of waters of hydration, e.g., monohydrate, dihydrate, trihydrate, tetrahydrate, pentahydrate, hexahydrate, heptahydrate, octahydrate, nonahydrate, etc.

The reason a metal atom can accept bonds over and above the oxidation state of the metal is due to the nature of chelation. For example, at the α-amino group of an amino acid, the nitrogen contributes both lone-pair electrons used in the bonding to the metal. These electrons fill available spaces in the d-orbitals of the metal forming a coordinate covalent bond. Thus, a metal ion with a normal valence of +2 can be bonded by up to eight bonds when fully chelated. In this state, the unfilled orbitals in the metal can be satisfied by both bonding electrons from lone pair electrons as well as electrons from ionic species. The chelate can be completely satisfied by the bonding electrons and the charge on the metal atom (as well as on the overall molecule) can still be zero. As stated previously, it is possible that the metal ion be bonded to the carboxyl oxygen by either coordinate covalent bonds or ionic bonds. However, the metal ion can also be bonded to the α-amino group by coordinate covalent bonds only.

Raman spectroscopy is a spectroscopic technique that can be used to study vibrational, rotational, and other low-frequency modes in a molecular system. Raman scattering, also known as inelastic scattering, can occur when a molecule absorbs a photon of light followed by an emission of photon of light which is either more or less energetic than the energy of the absorption. This difference in energy (also called the Raman shift), provides information about the molecule or molecules present in a sample. Raman scattering is typically very weak and, as a result, very difficult to separate from the more intense inelastic scattered light from Raleigh scattering (elastic scattering).

As briefly described above, inelastic scattering (Raman scattering) occurs when light impinges upon a molecule and interacts with the electron cloud in the bonds between atoms of a molecule (absorption). When the light impinges upon the electron cloud, the cloud may experience some degree of deformation which also polarizes the molecule. The amount of deformation of the electron cloud relates to the polarizability of the molecule. Polarizability is the relative tendency of the electron cloud of an atom to be distorted from its normal shape by the presence of a nearby ion or dipole—that is, by an external electric field. The electronic polarizability α is defined as the ratio of the induced dipole moment of an atom to the electric field that produces this dipole moment. expressed by the equation: p=αE.

The degree to which the electron cloud (bond) deforms between atoms in a molecule is directly proportional to the intensity and frequency of a Raman shift detectable using Raman spectroscopy. The absorption of a photon of light by the electron cloud in the molecule results in an electron attaining an excited state. After some finite period, the electron returns to a lower energy level and a photon of light is released. The lower energy level may be any number of different vibrational modes. When the electron returns to a ground vibrational state, a Stokes shift can occur. When the electron returns to a vibrational state higher than the ground state (such as a vibrational mode higher than the ground state before absorption), an anti-Stokes shift can occur. In some embodiments, Raman spectroscopy may be used to examine a vibrational spectrum spanning a range of about 3700 to about 100 cm⁻¹ and in some embodiments from about 500 to about 2000 cm⁻¹.

While Raman, as well as infrared (IR), spectroscopy can be used to identify and characterize chelates, the combination of both spectroscopic techniques can give a better evaluation of a molecule. IR and Raman spectroscopy differ in their respective techniques by the means which photonic energy is transferred from an instrument to the molecules in a sample. The molecular vibrational frequencies observed by both techniques are often similar, but the vibrational band intensities may differ (sometimes markedly so) because of the different excitation mechanisms and quantum selection rules. These differences can be representative of the different types of bonds in a molecule.

Vibrations initiated by IR can give rise to a molecular dipole change as the molecule contorts, i.e., the molecules can resonate with electromagnetic radiation of the same frequency. Because the vibrational frequencies of most molecules are similar to those found in the mid-infrared range, absorption transfers energy into the molecule causing it to vibrate more violently, thus giving rise to an IR spectrum. Vibrations initiated by Raman spectroscopy give rise to changes in polarizability simultaneously as the molecules vibrates and Raman scattering occurs.

Molecular bonds that end to have more ionic characteristics give rise to large bands observable in the IR spectrum, while these vibrations are weaker or undetectable in a Raman spectrum. Bonds that exhibit a more covalent, yet polarizable nature give rise to larger discernable bands in a Raman spectrum, while these vibrations are weaker or non-existent in the IR spectrum.

Samples can be prepared for Raman spectroscopic analysis by homogenizing a sample with a mortar and pestle and placing it in an sampling tube such as an NMR tube. Analysis can be performed by scanning from a variety of frequency ranges including 3700-100 cm⁻¹, as Raman shift, on a Raman spectrometer such as a Thermo-Nicolet FT-Raman Module Spectrometer. Analysis of the spectrum can include examination of frequencies associated with particular atomic and molecular bonds associated with chelate structures. When the chelate of interest includes one or more amino acids, the amine and carboxyl moieties of the amino acid may be examined. A change in the amine moiety in the zwitterionic amino acid from the NH₃ to the NH₂ configuration is of particular interest as the structure change is observed as a broad peak shift in IR where it is observable as a narrower separate peak in Raman. This as well as other molecular changes apparent in the Raman spectrum can identify the chelate structure and even quantitatively measure the amount of chelation.

Furthermore, larger proteinaceous amino acids and ligands may be characterized in the same manner as the simpler amino acid samples. Proteinaceous amino acids share an identical backbone which can participate in the chelate formation, namely an amine moiety in the alpha position relative to the carboxyl moiety.

The techniques described herein may be used to assure quality control for the production of a variety of metal ligand complexes including amino acid chelates. The techniques include examination of samples with metal ligand complexes using infrared spectroscopy, Raman spectroscopy and examination with both infrared and Raman spectroscopy.

Zinc bisglycinate chelate has been characterized using x-ray crystallography and mid-range FT-IR spectroscopy. A sample of zinc bisglycinate chelate so characterized was analyzed by FT-Raman spectroscopy. The vibrational spectrum obtained through IR spectroscopy was compared to the polarization spectrum obtained through Raman spectroscopy.

An initial characterization of glycine and zinc bisglycinate was carried out by obtaining Raman spectra for the two compounds. Several changes between the spectra were observed and are summarized in Table I.

| Band assignments as | | | |
|---|---|---|---|
| Raman shift (cm⁻¹) | | | |
| ZnGly₂ | Glycine | Bond | Observation |
| 3272 | | NH₂ | Only observable in a |
| | | | chelate |
| 2983, 2941 | 3007, 2972 | CH₂ | Asymmetric doublet shift |
| 1608, 1574 | 1668, 1631 | COO | Asymmetric doublet shift |
| | 1568 | NH₃ | Absent in chelate |
| 1436, 1414 | 1456, 1440 | CH₂ | Scissor doublet shift |
| | 1409 | COO | Not apparent in chelate |
| 1344, 1312 | 1324 | CH₂ | Changes to doublet |
| | 1136 | NH₃ | Absent in chelate |
| 1059 | 1035 | CN | Shift |
| 912 | 892 | CC | Shift |
| 581, 534, | | MO, MN | Peaks in chelate |
| 510, 469 | | | relating to metal bond |
| | 496 | COO or NH₃ | Absent in chelate |

An initial characterization of zinc bisglycinate was carried out by obtaining both Raman and IR spectra for the two compounds. Several characteristics from the spectra were observed and are summarized in Table II.

Observable difference

| Peaks of Interest | Raman | IR |
|---|---|---|
| NH₂ | Single sharp peak | Broad bands |
| CH₂ | Large response | Small response |
| COO⁻ | Small, sharp doublet | Large broad band |
| CN | Small, sharp band | Large sharp band |
| Chelate peaks | Small response, | Large broad bands |
| | sharp bands | |

The differences noted in Tables I and II demonstrate that Raman spectroscopy may be successfully used to characterize a chelate. Furthermore, both Raman spectroscopy and IR spectroscopy may be used together to characterize a chelate. Notable differences between the unbound glycine and the chelated glycine include peaks associated with the amine and carboxyl functional groups.

The techniques discussed above for the characterization of zinc bisglycinate can be applied to dry powdered samples.

In another example, the Raman spectra of a manganese bisglycinate chelate was obtained and compared to free glycine. The two spectra showed a change at the NH₂ stretch at 3270 cm⁻¹ and the chelate metal bonds in the 500 cm⁻¹ region.

In still another example, the Raman spectra of zinc aspartate and aspartic acid were obtained and compared. The two spectra showed a change at the NH₂ stretch at 3270 cm¹ and the chelate metal bonds in the 500 cm⁻¹ region.

In another aspect, Raman spectroscopy may be used to evaluate the reaction kinetics of a chelation reaction. For example, observing zinc bisglycinate reaction kinetics has been problematic because the molecule has no known chromophore. Likewise observing other chelate reaction kinetics has also been difficult. However, reaction kinetics of chelate reactions, such as zinc bisglycinate, can be achieved by observing samples with FT-Raman spectroscopy in aqueous and non-aqueous samples.

Using the FT-Raman spectrophotometer to determine zinc bisglycinate reaction kinetics, experimental data was collected at 15 second time intervals after contacting various amino acids with metal ligands to affect chelate synthesis. Changes in several frequencies associated with structure were monitored over set time intervals. Observations from these intervals permits the creation of a model for the reaction kinetics yielding a chelate. Observations included peak intensity or height at selected frequencies. When these observations are graphed, the rate order of the reaction may be determined. Other metal ligand complexes and their synthesis reaction kinetics may be examined such as non-amino acid ligands.

For example, the reaction kinetics of zinc bisglycinate formation were observed. Samples were measured using a Nicolet FT-Raman module with a high energy Nd: YVO₄ laser with a range of power settings from 0.5 to 2.0 watts to generate Raman scatter at 15 second intervals after the reaction was initiated. The reaction mixture was stoichiometrically balanced to model the above reaction. One mole of zinc oxide, dry mixed with two moles of glycine (Gly) was thoroughly ground and mixed using a mortar and pestle. 60 mg of the ZnO/Gly dry-mix was placed into a 5 mm×25 mm glass tube. 35 mg of anhydrous sodium sulfite was placed on top of the ZnO/Gly dry-mix as a wick to provide a delay in the initial reaction slowing the flow of water into the reaction mixture. The Raman spectrometer was then started and 35 μL of water was added by micropipette to the glass tube. A Raman spectrum was obtained at 4 second intervals for 60 second over a spectrum range of 4000 to 100 cm⁻¹. A total of fifteen Raman spectra were obtained over the 60 second experiment.

Peak heights for five peaks measured in each of the fifteen spectra were noted and plotted. The five peaks corresponded to the identified functional groups in the Raman spectrum of free glycine: 1324, 1136, 1107, 1035, and 892 cm⁻¹. Rate constants and reaction order with respect to the chosen peaks were calculated using linear regression. The R-squared values for data associated with the five peaks of interest were calculated and are summarized in Table III.

| Peak (cm⁻¹) | R-squared value |
|---|---|
| 1324 | 0.9231 |
| 1136 | 0.8594 |
| 1107 | 0.6533 |
| 1035 | 0.7840 |
| 892 | 0.9036 |

As seen from the R-squared values in Table III, the kinetic rates do not appear to follow first order kinetics. The data was recalculated with a second order model kinetic rate without improvement to the R-squared values. Close examination of the curves exhibit different results for reach frequency (i.e., r different part of the amino acid molecule). Resemblances were observed comparing the data curves among themselves. For example, the peak intensities at 1324 cm⁻¹ corresponding to a carbon-methylene way had nearly the same slope when compared to the peak intensities at 892 cm⁻¹ for a carbon-carbon stretch. Within the glycine molecule, the carbon-carbon bonds are not exchanging electrons and forming new bonds as the nitrogen or oxygen atoms do upon chelate formation. A difference is revealed when the plotted curves of the carbonyl, amine, and the carbon-nitrogen peaks are compared to each other. The peaks disappear in what appears to be a stepwise or incremental fashion as the reaction progresses, suggesting a zero order kinetic reaction.

New peaks in the Raman spectra were observed during reaction include 1063 and 915 cm⁻¹.

We claim:

1. A method of assaying for the presence or absence of a metal-ligand chelate in a sample comprising:
irradiating with electromagnetic energy a sample containing a ligand;
obtaining a first Raman electromagnetic spectrum of the ligand;
contacting the ligand in the sample with a metal;
obtaining a second Raman electromagnetic spectrum;
comparing the first Raman electromagnetic spectrum with the second Raman electromagnetic spectrum to detect a change.

2. The method according to claim 1, wherein the metal is selected from the group consisting of: boron, calcium, chromium, cobalt, copper, iron, magnesium, manganese, potassium, selenium, vanadium and zinc, and the ligand is selected from the group consisting of: alanine, aspartic acid, cysteine, glutamic acid, glycine, histidine, isoleucine, leucine, lysine, methionine, phenylalanine, serine, threonine, tryptophan, tyrosine and valine.

3. The method according to claim 1, wherein the ligand is selected from the group consisting of: alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine.

4. The method according to claim 1, wherein at least one of the first and second Raman electromagnetic spectra have a wavelength peak corresponding to one or more functional groups selected from the group consisting of: amine, hydroxyl, thiol, and carboxylic acid.

5. The method according to claim 1, further comprising:
obtaining a first IR electromagnetic spectrum of the sample;
obtaining a second IR electromagnetic spectrum;
comparing the first IR electromagnetic spectrum with the second IR electromagnetic spectrum to detect a change.

6. The method according to claim 5, further comprising analyzing both the Raman and IR spectral changes to characterize a chelate.

7. The method according to claim 6, further comprising calculating a reaction rate.

8. A method for monitoring chelation synthesis reactions comprising:
irradiating a sample containing a free metal and a free ligand with electromagnetic energy;
obtaining a Raman spectrum of the sample;
reacting the free metal with the free ligand to form a metal-ligand chelate;
analyzing the Raman spectrum to detect one or more of the free metal, the free ligand, and metal-ligand chelate, the presence or absence of which is indicative of the progress of chelation synthesis.

9. The method according to claim 8, wherein the free metal is selected from the group consisting of: boron, calcium, chromium, cobalt, copper, iron, magnesium, manganese, potassium, selenium, vanadium and zinc, and the free ligand is selected from the group consisting of: alanine, aspartic acid, cysteine, glutamic acid, glycine, histidine, isoleucine, leucine, lysine, methionine, phenylalanine, serine, threonine, tryptophan, tyrosine and valine.

10. The method according to claim 8, wherein the free ligand is selected from the group consisting of: alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine.

11. The method according to claim 8, wherein at least one of the first and second Raman electromagnetic spectra have a wavelength peak corresponding to one or more functional groups selected from the group consisting of: amine, hydroxyl, thiol, and carboxylic acid.

12. The method according to claim 8, further comprising:
obtaining an IR electromagnetic spectrum of the sample;
analyzing the IR spectrum to detect one or more of the free metal, the free ligand, and metal-ligand chelate, the presence or absence of which is indicative of the progress of chelation synthesis.

13. The method according to claim 12, further comprising analyzing the Raman and IR spectra for changes to characterize a chelate.

14. The method according to claim 13, further comprising calculating a reaction rate.

15. A method of assaying for the presence or absence of a metal-ligand chelate in a sample comprising:
irradiating with electromagnetic energy a sample suspected of containing one or more of a free ligand and a metal-ligand chelate;
obtaining a Raman electromagnetic spectrum of the sample;
analyzing the Raman electromagnetic spectrum to detect one or more of a free metal, the free ligand, and the metal-ligand chelate, the presence or absence of which is indicative of the metal-ligand chelate.

16. The method according to claim 15, wherein the metal is selected from the group consisting of: boron, calcium, chromium, cobalt, copper, iron, magnesium, manganese, potassium, selenium, vanadium and zinc, and the ligand is selected from the group consisting of: alanine, aspartic acid, cysteine, glutamic acid, glycine, histidine, isoleucine, leucine, lysine, methionine, phenylalanine, serine, threonine, tryptophan, tyrosine and valine.

17. The method according to claim 15, wherein the ligand is selected from the group consisting of: alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine.

18. The method according to claim 15, wherein the Raman electromagnetic spectra have a wavelength peak corresponding to one or more functional groups selected from the group consisting of: amine, hydroxyl, thiol, and carboxylic acid.

19. The method according to claim 15, further comprising:
obtaining an IR electromagnetic spectrum of the sample;
analyzing the IR electromagnetic spectrum to detect one or more of the free metal, the free ligand, and the metal-ligand chelate, the presence or absence of which is indicative of the metal-ligand chelate.

20. The method according to claim 19, further comprising analyzing both the Raman and IR spectral changes to characterize a chelate.

21. The method according to claim 20, further comprising calculating a reaction rate.
